(19) United States
(12) Patent Application Publication
(10) Pub. No.: US 2025/0241866 A1
(43) Pub. Date: Jul. 31, 2025

(54) STRUCTURAL DESIGN OF FLEXIBLE DISKS TO MODULATE MECHANOTRANSDUCTION MEDIATED BY INTEGRIN RECEPTORS

(71) Applicant: Jichul KIM, Changwon-si (KR)

(72) Inventor: Jichul KIM, Changwon-si (KR)

(21) Appl. No.: 18/854,992

(22) PCT Filed: Jun. 6, 2022
(86) PCT No.: PCT/KR2022/007963

§ 371 Date: Oct. 8, 2024

(51) Int. Cl. A61K 9/51

(52) U.S. Cl. CPC A61K 9/5169

(57) ABSTRACT

Substances in the form of flexible disks to modulate mechanotransduction mediated by integrins were designed. Two main ideas for the design are as follows. First, the radius of the disk can be prescribed to specify the radius of mechanically perturbed integrin clusters. By using disks with a greater radius value, the vinculin binding site activation vs. force relation can be amplified and shifted to the positive direction along the input axis. Second, by recruiting a certain integrin whose affinity values to talins are higher than those of other integrins into the cluster, the radial distance from the center of clusters to the point where the membrane tightly adheres to the cytoskeleton can be decreased. This results in the increase of the maximum slope of the vinculin binding site activation vs. force relation. The design can be used in the development of drugs for diseases associated with dysfunctions in integrin-mediated systems.

### TECHNICAL FIELD

This work introduces the structural design of substances to modulate integrin-mediated mechanical signal transduction (or mechanotransduction).

### BACKGROUND ART

Integrin receptors are adhesion proteins found in various mammalian cells (Gilcrease, 2007). Dysfunctions in the integrin-mediated system results in various pathological and aging phenomena including diabetes, cancer metastasis, micro-vein occlusion, and skin aging (Has et al., 2012; Makrantonaki & Zouboulis, 2007; Parnaud et al., 2006; Ramsay, Marshall, & Hart, 2007; Roth, Podesta, Stepp, Boeri, & Lorenzi, 1993).

Mechanobiological approaches can be considered to cure diseases associated with dysfunctions in the integrin-mediated system. In particular, drugs to modulate characteristic mechanobiological responses mediated by integrins can be designed because the curve shape of the response can be linked to certain disease states.

Cellular activities can be changed when mechanical inputs are applied to integrin receptors in cell surfaces. The process known for mechanical signal transduction (or mechanotransduction) can be defined by cellular activity vs. mechanical input relations. For example, YAP nuclear translocation vs. substrate rigidity relations that show the sigmoidal function shape in the semi-log scale were investigated (Elosegui-Artola et al., 2016). Modifications in the cellular activity vs. mechanical input curve shape were identified for cells in physiologically less relevant conditions (Elosegui-Artola et al., 2016). Therefore, it is important to investigate methods to rescue the normal cellular activity vs. mechanical input relation for those cells.

According to a recent work, among many possibilities across the cell membrane, the mechanical response of lipid membranes explains the mechanical response and mechanotransduction mediated by integrins (Kim, 2021). The work used a theoretical lipid membrane model validated by experiments (Kim, 2020) and biological data on integrin adaptor talin proteins (Calderwood et al., 1999; Del Rio et al., 2009; Goult et al., 2013; Ringer et al., 2017; Yao et al., 2016) (also see references in (Kim, 2021)). The work identified vinculin binding site (VBS) activation vs. force relations at single integrin cluster levels that also show the sigmoidal function shape and serve as an indicator for the integrin mechanotransduction process (Kim, 2021). The work identified two important membrane boundary parameters that vary the curve shape of the VBS activation vs. force relation (Kim, 2021).

The first is the radius of mechanically perturbed single integrin clusters, defined by r_{ca}[FIG. 1]. According to the work, the amplitude of the VBS activation vs. force relation can be increased, and the response can be shifted to the positive direction along the input axis by increasing the r_{ca} value (Kim, 2021).

The second is the radial distance from the center of mechanically perturbed single integrin clusters to the point where the membrane tightly adheres to the cytoskeleton. This radial distance is defined by r_{ct}[FIG. 1]. By decreasing the r_{ct} value, the maximum slope of the VBS activation vs. force relation can be increased. In addition, by decreasing r_{ct}, the ratio between the length of the steep slope region in the output axis and the total amplitude of the response can be increased (Kim, 2021).

In this work, flexible disk-like structures were designed to modulate the r_{ca} and r_{ct} values [FIG. 1], and thus to modulate the mechanotransduction mediated by integrins at the single integrin cluster level.

To specify the r_{ca} value, the radius of the disk defined by r_{fd} is prescribed. The disk binds the single integrin cluster to the extracellular matrices (ECMs). Therefore, with forces applied on the cluster, the r_{ca} value can be similarly defined with the f_{rd} value. To modulate the r_{ct} value, different disks can be functionalized to recruit different integrins. By using the disk to recruit a certain integrin whose affinity values to talins are higher than those of other integrins into the cluster, the r_{ct} value can be decreased as more integrin-talin complexes can be separated apart from the cluster to limit the perturbation of membranes (Kim, 2021) [FIG. 2].

To recruit a certain integrin, a certain integrin-binding peptide or certain integrin binding peptides can be functionalized for the flexible disk. There are several peptides introduced. For example, α₃β₁, α₅β₁, αᵥβ₁, αᵥβ₃, αᵥβ₅, αᵥβ₆, and α_{IIb}ß₃ interact with the RGD peptide. α₄ß₁ interacts with the REDV peptide. α₄β₁, α₄β₇, and α₉β₁ interact with the LDV peptide. α₄β₁ and α₂β₁ interact with the GFOGER peptide. α₂β₁ interacts with the DGEA peptide. α₃β₁ interacts with the IKLLI peptide. α₂β₁ interacts with the EF1zz peptide. α₅β₁ interacts with the CRRETAWAC peptide (Heath & Cooper, 2017). To synthesize the disk-like structure by considering the peptide functionalization, methods used in the development of multivalent integrin binding peptides can be utilized (Garanger, Boturyn, Coll, Favrot, & Dumy, 2006). Data that demonstrate variations on affinity values between different integrin β subunits and talins also support the idea employed in this design (Anthis et al., 2009). A report demonstrated catch bonds between the integrin and its ligand (Kong, García, Mould, Humphries, & Zhu, 2009).

### SUMMARY OF INVENTION

The design of nanoscale substances in the form of flexible disks is proposed.

In this design, one surface of the disk is functionalized with a certain integrin binding peptide or certain integrin binding peptides and the other surface is functionalized with a certain extracellular matrix (ECM) binding peptide or certain ECM-binding peptides.

The flexible disk can be designed to show the catch bond or the slip bond for the interaction between the integrin and the integrin-binding peptide(s) as well as for the interaction between the ECM and the ECM-binding peptide(s).

### Technical Problem

Single cluster level substances or drugs to modulate mechanotransduction mediated by integrins are required to cure and prevent diseases associated with dysfunctions in the integrin-mediated system.

### Solution to Problem

Substances or drugs to modulate the r_{ca} and r_{ct} values can be developed. In this reason, flexible disks that bind single integrin clusters to ECMs can be designed [FIG. 1].

The radius of the disk r_{fd} is prescribed to specify the r_{ca} value. Then, the amplitude of the VBS activation vs. force relation can be increased by using the disk that shows a greater r_{fd} value. In addition, the maximum slope point of the response can be shifted to the positive direction of the input axis by using the disk with an increased r_{fd} value. Similarly, the amplitude can be decreased and the response can be shifted to the negative direction by using a decreased r_{fd} (Kim, 2021).

When the cluster is pulled, several integrins coupled with talins can be separated apart from the cluster and slide in the membrane to reduce forces on the talins. As illustrated in [FIG. 2] the process can contribute to reduce the rat value. In this process, if the number of the talin-coupled integrin in the mechanically perturbed cluster is increased, the number of the integrin-talin complex that can be separated apart from the cluster can be also increased. Therefore, a certain integrin that shows higher affinity values to talins can be recruited by functionalizing the surface of the disk with a certain integrin binding peptide or certain integrin binding peptides to decrease r_{ct}.

By decreasing r_{ct}, the maximum slope of the VBS activation vs. force relation can be increased (Kim, 2021). In addition, by decreasing r_{ct}, the ratio between the length of the steep slope region in the output axis and the total amplitude of the response can be increased (Kim, 2021). Similarly, a certain integrin that shows smaller affinity values to talins can be recruited into the cluster, when larger r_{ct} is required. Finally, it is also important to note that one disk type can recruit multiple integrin cluster types.

### Advantageous Effects of Invention

The design can be used to develop drugs for diseases associated with dysfunctions in the integrin-mediated system. In addition, the concepts of the design fit well with the paradigm of the precise and personalized medicine.

### BRIEF DESCRIPTION OF DRAWINGS

FIG. 1 illustrates how the flexible disk interacts with mechanically perturbed integrin clusters and ECMs. How each surface of the disk is functionalized is also described in [FIG. 1]. Three important radius values i.e., r_{ca}, r_{ct}, and r_{fd}, are defined. For simplicity, talins are not shown.

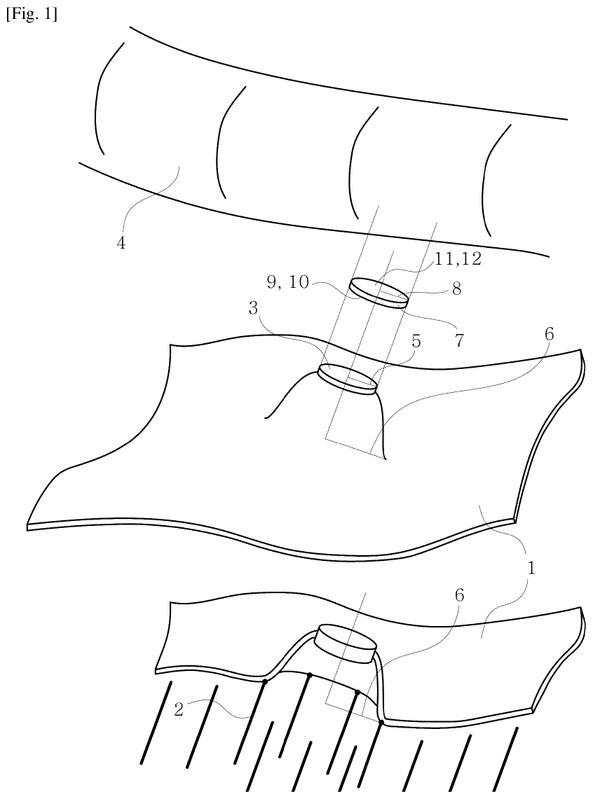

FIG. 2 demonstrates how r_{ct} can be decreased when forces are applied on integrin clusters and talins. From a configuration that shows a larger r_{ct} value (top), several integrins coupled with talins can be separated apart from the cluster to reduce the tension in the talins (middle). This results in a configuration that shows a smaller r_{ct} value (bottom). The processes illustrated in the top, middle, and bottom panels can be repeated until a certain r_{ct} value can be defined.

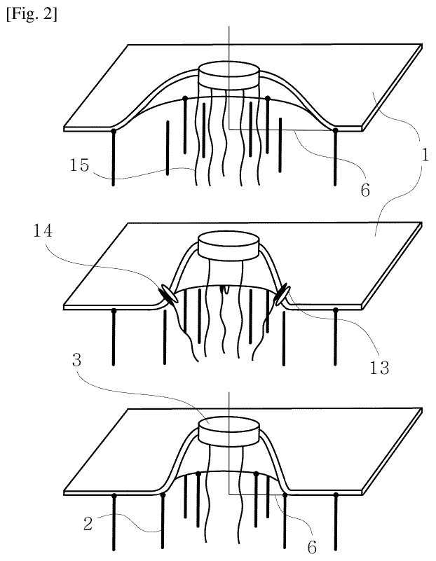

### DESCRIPTION OF EMBODIMENTS

The design can be used in the development of substances or drugs for biological researches or cosmetic or therapeutic treatments on diseases associated with dysfunctions in the integrin-mediated system.

### EXAMPLES

The ideas employed in this design can be used in the development of substances or drugs for biological researches or cosmetic or therapeutic treatments on diseases associated with dysfunctions in the non-integrin-mediated system.

### INDUSTRIAL APPLICABILITY

The design can be used in the development of substances or drugs for biological researches or cosmetic or therapeutic treatments on diseases associated with dysfunctions in the integrin-mediated system.

### REFERENCE SIGNS LIST

1: Cell membranes
2: Cytoskeletons
3: Mechanically perturbed integrin clusters
4: Extracellular matrices (ECMs)
5: The radius of mechanically perturbed single integrin clusters, r_{ca}
6: The radial distance from the center of the mechanically perturbed integrin cluster to the point where the membrane tightly adheres to the cytoskeleton, r_{ct}. The tight adhesion is indicated with black dots.
7: Substances in the form of flexible disks that bind the single integrin cluster to ECMs
8: The radius of the flexible disk r_{fd} to prescribe r_{ca}
9: The integrin binding surface of the disk. The surface is functionalized with a certain integrin-binding peptide or certain integrin-binding peptides.
10: The surface can be designed to show the catch bond or the slip bond for the interaction between the integrin and the integrin-binding peptide(s).
11: The ECM binding surface of the disk. The surface is functionalized with a certain ECM-binding peptide or certain ECM-binding peptides.
12: The surface can be designed to show the catch bond or the slip bond for the interaction between the ECM and the ECM-binding peptide(s).
13: Integrin α subunits
14: Integrin β subunits
15: Talins

### REFERENCE TO DEPOSITED BIOLOGICAL MATERIAL

N/A

### Sequence Listing Free Text

N/A

### Citation List

N/A

### PATENT LITERATURE

PTL 1: N/A

### NON-PATENT LITERATURE

Anthis, N. J., Haling, J. R., Oxley, C. L., Memo, M., Wegener, K. L., Lim, C. J., . . . . Campbell, I. D. (2009). β integrin tyrosine phosphorylation is a conserved mechanism for regulating talin-induced integrin activation. Journal of Biological Chemistry, 284(52), 36700-36710.
Calderwood, D. A., Zent, R., Grant, R., Rees, D. J. G., Hynes, R. O., & Ginsberg, M. H. (1999). The talin head domain binds to integrin β subunit cytoplasmic tails and regulates integrin activation. Journal of Biological Chemistry, 274(40), 28071-28074.
Del Rio, A., Perez-Jimenez, R., Liu, R., Roca-Cusachs, P., Fernandez, J. M., & Sheetz, M. P. (2009). Stretching single talin rod molecules activates vinculin binding. Science, 323(5914), 638-641.
Elosegui-Artola, A., Oria, R., Chen, Y., Kosmalska, A., Pérez-González, C., Castro, N., . . . . Roca-Cusachs, P. (2016). Mechanical regulation of a molecular clutch defines force transmission and transduction in response to matrix rigidity. Nature cell biology, 18(5), 540-548.
Garanger, E., Boturyn, D., Coll, J.-L., Favrot, M.-C., & Dumy, P. (2006). Multivalent RGD synthetic peptides as potent α V β 3 integrin ligands. Organic & biomolecular chemistry, 4(10), 1958-1965.
Gilcrease, M. Z. (2007). Integrin signaling in epithelial cells. Cancer letters, 247(1), 1-25.
Goult, B. T., Xu, X.-P., Gingras, A. R., Swift, M., Patel, B., Bate, N., . . . Volkmann, N. (2013). Structural studies on full-length talin1 reveal a compact auto-inhibited dimer: implications for talin activation. Journal of structural biology, 184(1), 21-32.
Has, C., Sparta, G., Kiritsi, D., Weibel, L., Moeller, A., Vega-Warner, V., . . . . Esser, P. (2012). Integrin α3 mutations with kidney, lung, and skin disease. New England Journal of Medicine, 366(16), 1508-1514.
Heath, D. E., & Cooper, S. L. (2017). The development of polymeric biomaterials inspired by the extracellular matrix. Journal of Biomaterials science, Polymer edition, 28(10-12), 1051-1069.
Kim, J. (2020). Probing nanomechanical responses of cell membranes. Scientific reports, 10(1), 1-11.
Kim, J. (2021). A possible molecular mechanism for mechanotransduction at cellular focal adhesion complexes. Biophysical Reports, 1(1), 100006.
Kong, F., García, A. J., Mould, A. P., Humphries, M. J., & Zhu, C. (2009). Demonstration of catch bonds between an integrin and its ligand. Journal of Cell Biology, 185 (7), 1275-1284.
Makrantonaki, E., & Zouboulis, C. (2007). Molecular mechanisms of skin aging: state of the art. Annals of the New York Academy of Sciences, 1119(1), 40-50.
Parnaud, G., Hammar, E., Rouiller, D. G., Armanet, M., Halban, P. A., & Bosco, D. (2006). Blockade of β1 integrin-laminin-5 interaction affects spreading and insulin secretion of rat β-cells attached on extracellular matrix. Diabetes, 55(5), 1413-1420.
Ramsay, A. G., Marshall, J. F., & Hart, I. R. (2007). Integrin trafficking and its role in cancer metastasis. Cancer and Metastasis Reviews, 26(3), 567-578.
Ringer, P., Weißl, A., Cost, A.-L., Freikamp, A., Sabass, B., Mehlich, A., . . . Grashoff, C. (2017). Multiplexing molecular tension sensors reveals piconewton force gradient across talin-1. Nature methods, 14 (11), 1090-1096.
Roth, T., Podesta, F., Stepp, M. A., Boeri, D., & Lorenzi, M. (1993). Integrin overexpression induced by high glucose and by human diabetes: potential pathway to cell dysfunction in diabetic microangiopathy. Proceedings of the National Academy of Sciences, 90 (20), 9640-9644.
Yao, M., Goult, B. T., Klapholz, B., Hu, X., Toseland, C. P., Guo, Y., . . . . Yan, J. (2016). The mechanical response of talin. Nature communications, 7(1), 1-11.

1. Substances in the form of flexible disks whose radial size is greater than 25 nm, functionalized with a certain integrin-binding peptide or certain integrin-binding peptides on their one surface, and functionalized with a certain ECM-binding peptide or certain ECM-binding peptides on the other surface.

2. Regarding the claim 1, substances designed to show the catch bond for the interaction between the integrin and the integrin-binding peptide(s) and the catch bond for the interaction between the ECM and the ECM-binding peptide(s).

3. Regarding the claim 1, substances designed to show the slip bond for the interaction between the integrin and the integrin-binding peptide(s) and the slip bond for the interaction between the ECM and the ECM-binding peptide(s).

4. Regarding the claim 1, substances designed to show the catch bond for the interaction between the integrin and the integrin-binding peptide(s) and the slip bond for the interaction between the ECM and the ECM-binding peptide(s).

5. Regarding the claim 1, substances designed to show the slip bond for the interaction between the integrin and the integrin-binding peptide(s) and the catch bond for the interaction between the ECM and the ECM-binding peptide(s).
